(19) United States
(12) Patent Application Publication
(10) Pub. No.: US 2006/0079844 A1
(43) Pub. Date: Apr. 13, 2006

(54) Needle apparatus

(76) Inventors: Maxwell Edmund Whisson, Subiaco (AU); Dean Brian Prestidge, Mullaloo (AU)

Correspondence Address: BACHMAN & LAPOINTE, P.C., 900 CHAPEL STREET, SUITE 1201, NEW HAVEN, CT, 06510, US

(21) Appl. No.: 10/530,299

(22) PCT Filed: Oct. 7, 2003
(86) PCT No.: PCT/AU03/01312

§ 371 Date: Apr. 5, 2005

(30) Foreign Application Priority Data
Oct. 7, 2002 (AU) 2002951827
Nov. 29, 2002 (AU) 2002953025

(51) Int. Cl. A61M 5/178

(52) U.S. Cl. 604/164.08

(57) ABSTRACT

A needle apparatus having a thin walled sleeve and a tubular needle closely engaged by the sleeve. The needle has a sharp point which initially extends from the sleeve. The needle and sleeve are mounted to a hub and are longitudinally moveable relative to one another. The needle is arranged to pierce tissue and the sleeve enters the incision formed by the needle. The needle may then be withdrawn from the tissue by applying traction to the hub. The sleeve is retained in place in the tissue as a result of pressure applied radially by surrounding tissue

### BRIEF DESCRIPTION OF THE INVENTION

The present invention relates to needle apparatus

### SUMMARY OF THE INVENTION

In accordance with one aspect of the present invention there is provided A needle apparatus characterised by a thin walled sleeve having a proximal end and a distal end, a tubular needle having a distal end and a proximal end, the needle having a sharp point at the distal end thereof, the needle being closely engaged by the sleeve, the needle and the sleeve being longitudinally moveable relative to one another between a first position at which the needle extends from the sleeve and a second position at which the sharp point is located within the sleeve, the apparatus further comprising a hub in which the needle is fixedly mounted adjacent the proximal end of the needle, the needle extending from the hub so that the distal end thereof is located externally of the hub, the sleeve having the proximal end thereof located within the hub and being longitudinally slidably mounted relative to the hub, the sleeve extending from the hub so that the distal end thereof is located externally of the hub, the needle apparatus being arranged to pierce tissue when the sleeve and the needle are in the relative first position and being such that after the tissue has been pierced the needle is arranged to be withdrawn from the tissue whilst the sleeve remains in situ in the tissue as a result of the pressure applied radially by surrounding tissue.

### DESCRIPTION OF THE DRAWINGS

The present invention will now be described, by way of example, with reference to the accompanying drawings, in which:

FIG. 1 is a longitudinal sectional view of a first embodiment of a needle apparatus in accordance with the present invention in a first position ready for use;

FIG. 2 is a view similar to FIG. 1 with the needle apparatus in a second position;

FIG. 3 is a longitudinal sectional view of a second embodiment of the needle apparatus in accordance with the present invention in a first position ready for use;

FIG. 4 is a view similar to FIG. 3 with the needle apparatus in a second position;

FIG. 5 is a view similar to FIG. 4 with a catheter introducer portion of the needle apparatus separated from the remainder thereof.

### DETAILED DESCRIPTION OF THE INVENTION

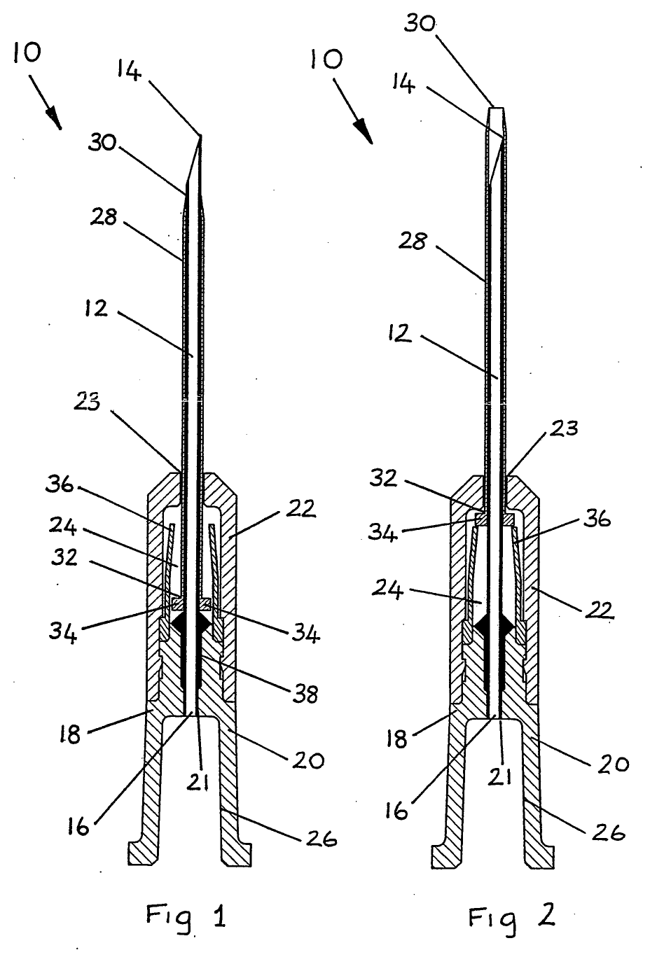

In FIGS. 1 and 2 of the accompanying drawings there is shown a needle apparatus 10 comprising a needle 12 which has a distal end 14 which has a sharp point. The needle 12 also has a proximal end 16.

The needle 12 is mounted in a hub 18 adjacent the proximal end 16 of the needle 12. As will be described the needle 12 is fixed to the hub 18 for movement therewith. The hub 18 is formed in two parts. A first part 20 of the hub 18 has a longitudinal central aperture 21 therein in which is received the needle 12 adjacent the proximal end 16. A second part 22 of the hub 18 is mounted about the needle 12 by means of a central aperture 23. The second part 22 is generally U-shaped in cross-section as seen in FIGS. 1 and 2. An open end of the U-shape is engaged with the first part 20 to form a hollow chamber 24.

Further, the first part 20 is formed with a means 26 for connection to a tube or a container or a blood vacuum supply system or a syringe. As shown in the drawings the first part 20 of the hub 18 is formed with a female Luer taper 26.

A sleeve 28 is mounted about the needle 12. As will be described, the sleeve 28 is arranged for longitudinal movement relative to the hub 18 and the needle 12. The sleeve 28 may be formed of flexible material such as a plastics material or it may be formed of relatively rigid material such as a metallic material. As shown, the sleeve 28 has a distal end 30 adjacent the distal end 14 of the needle 12. Further, the sleeve 28 extends through the central aperture 23 in the second part 22 of the hub 18 and into the chamber 24 to terminate in a proximal end 32.

Adjacent the proximal end 32 the sleeve 28 is formed with a laterally outwardly extending projection 34. In the embodiment shown the projection 34 is a disc but the projection 34 could take many forms. In fact, the projection 34 could be any shape with a radially or laterally extending surface.

Further, a plurality of resilient fingers 36 are mounted to a distal end of the first part 20 of the hub 18 so as to project into the periphery of the chamber 24 as shown. In the condition shown in FIG. 1 the fingers 36 extend about and beyond the projection 34. It is envisaged that only a single finger 36 could be provided. Also, the or each finger 36 could engage an adjacent edge of the projection 34 in the position shown in FIG. 1 to assist in retaining the sleeve 28 in place.

As shown, the fingers 36 are biased inwardly towards the needle 12 at distal ends remote from the first part 20. At their distal ends the external spacing between the fingers is less than the lateral extent of the projection 34.

A stop member 38 is provided in the first part 20 and extends about the needle 12. The stop member 38 prevents movement of the projection 34 proximally away from the distal end 14 of the needle 12.

The sleeve 28 is dimensioned so as to be able to move axially relative to the needle 12 but also to move axially relative to the second part 22 of the hub 18 in the chamber 24.

Further, in the position shown in FIG. 1 the sleeve 28 is unlatched and does not have any positive means for preventing movement thereof relative to the hub 18 except for the minor pressure applied by the finger or fingers 36 and the frictional engagement with the hub 18 in the aperture 23.

The sleeve 28 has an internal surface which is smooth with low friction. The friction may be reduced further by application of a film of lubricant such as silicone oil between the needle 12 and the sleeve 28.

The sleeve 28 also has an external surface which is also preferably smooth. However, the external surface may be formed with an asymmetrical characteristic by modification of the surface microscopically such as by chemical etching or by making a number of very small ridges or incisions at an angle to an axis of the sleeve 28 so that resistance is greater for proximal movement of the sleeve 28 than for distal movement.

In use, the needle apparatus 10 is initially in the position shown in FIG. 1 with the distal end 14 of the needle projecting from the sleeve 28. The distal end 14 of the needle 12 is inserted into living tissue to form an incision. The sleeve 28 then enters the incision.

At this point the sleeve 28 is subjected to elastic pressure from the surrounding tissue. This pressure is resisted by the compressive strength of the cannula so that resistance to movement between the sleeve 28 and the needle 12 is not significantly increased by the pressure of the needle 12 and the sleeve 28 in the tissue.

The hub 18 is then moved manually proximally away from the distal end 30 of the sleeve 28. The sleeve 28 is retained in the incision by the elastic pressure from the tissue whilst the needle is retracted with the hub 18 towards the position shown in FIG. 2. As this movement occurs the fingers 36 move longitudinally relative to the projection 34 causing them to be displaced laterally and ride up over the projection 34. As the fingers 36 move to a proximal position relative to the projection 34 they move inwardly towards the needle 12 allowing their tips to engage the proximal surface of the projection 34 as shown in FIG. 2. This prevents retraction of the sleeve 28 relative to the needle 12.

It will be seen that the needle is only moved a relatively short distance such as about 2 to 3 mm. but in the condition shown in FIG. 2 the distal end 14 is within the sleeve 28 and is thus rendered safe. Further, the sleeve 28 can be used for fluid transfer from or to the living tissue in which it is inserted. It will be noted that the needle 12 remains in the fluid flow path at all times. Once the fluid transfer has been completed the sleeve 28 is typically withdrawn from the living tissue by further traction on the hub 18. The needle apparatus 10 may then be safely disposed of.

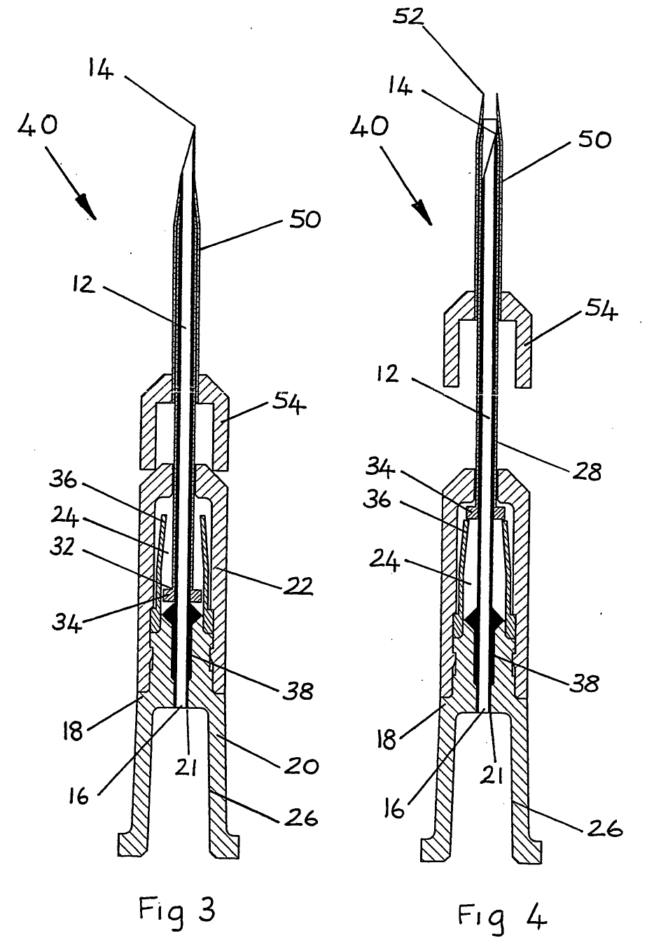

In FIGS. 3, 4 and 5 of the accompanying drawings there is shown a needle apparatus 40 which is in many respects similar to the needle apparatus 10. Like reference numerals denote like parts.

In the needle apparatus 40 there is additionally provided an outer sheath 50 which is in the form of a flexible sleeve or catheter. The sheath 50 fits closely on the outer surface of the sleeve 28. The sheath 50 is arranged to slide axially relative to the sleeve 28 and the resistance to axial movement is greater than the resistance to axial movement of the sleeve 28 on the needle 12 from the first position shown in FIG. 3 to the second position shown in FIG. 4. The sheath 50 is mounted to a hub 54. The sheath 50 is preferably a flexible plastics tube.

The sheath 50 has a distal end 52. Further, the sheath 50 preferably has a wall thickness which tapers downwardly towards the end 52. As shown in FIG. 3, in the initial condition of the needle apparatus 40, the distal end 52 of the sheath 50 is close to the distal end 30 of the sleeve 28.

Tissue pressure may not be significantly transmitted through the sheath 50 to the sleeve 28. The sheath 50 may be quite a tight fit on the sleeve 28 but an operator could readily pull the sleeve 28 and the needle 12 from the sheath 50.

The tapering of the wall thickness minimises a shoulder formed by open ends of the sleeve 28 and the sheath 50. This reduces hindrance of entry of the needle 12 into living tissue as described hereinabove.

The sheath 50 is a close fit on the outer surface of the sleeve 28. Closeness of fit and grip of the sheath 50 on the sleeve 28 is not significantly changed by tissue pressure. The force required to remove the sheath 50 from the sleeve 28 is very much greater than the force required to move the needle 12 proximally within the sleeve 28 so allowing it to reach the second position. This occurs when traction is applied to the hub 18 in the normal action of withdrawing the needle 12 from the tissue. In the initial motion of the hub 18, the needle 12 and the sleeve 28, the sleeve 28 does not move because of the tight friction fit with the sheath 50 and the sheath 50 is retained during this small movement by friction of the surrounding tissue acting on the outer surface of the sheath 50. The operator then applies a stronger traction when it is desired to withdraw the needle 12 from within the sheath 50, so leaving the sheath 50 in the blood vessel.

Thus, initial withdrawal of the needle 12 causes the needle apparatus to move to the second position shown in FIG. 4 in which the sleeve 12 projects beyond the needle tip 14.

In FIG. 4, the needle 12 has moved proximally relative to the sleeve 28 and the sheath 50. The sheath 50 is retained in place by radial pressure of the surrounding tissue. Alternatively, the sleeve 28 could be withdrawn relative to the sheath 50 manually by applying pressure to the hub 54 or straping the hub 54 to the skin such as by means of adhesive tape.

In the position shown in FIG. 4 the sheath 50 may be safely advanced into a vein or artery without puncturing the far wall of the vein or artery.

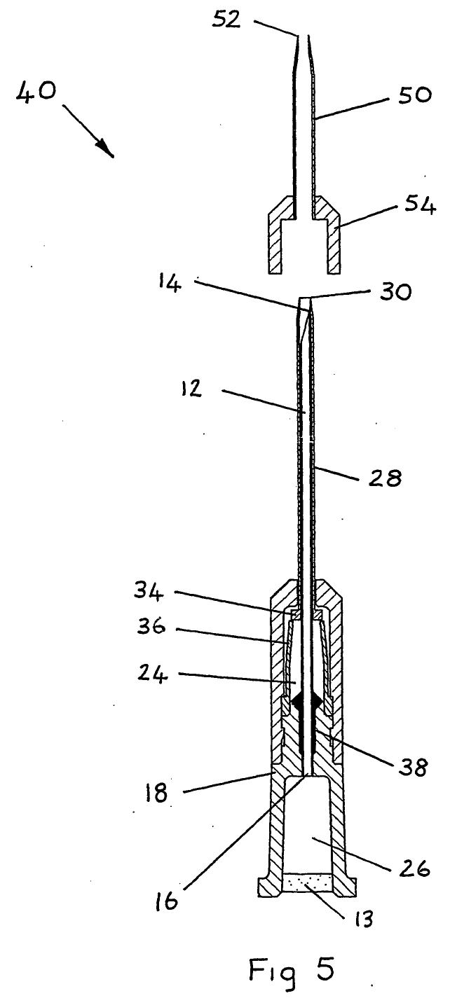

In FIG. 5, there is shown a third position in which the needle apparatus 10 has been withdrawn from the sheath 50 whilst the sheath 50 may remain in situ in a blood vessel.

As shown in FIG. 5, the Luer taper 26 may further comprise an air permeable disc 13 which is arranged to allow blood to enter the Luer taper 26 so that flash back can confirm the pressure of the needle in a blood vessel. The flow of blood through the disc 13 is restricted by the small pore size of the material of which it is constructed.

In the position shown in FIG. 5, the sheath 50 may be used in a number of different ways. For example, it may form an indwelling catheter for infusion of fluid into a blood vessel. However, the sheath 50 may also act as a guide for introduction of longer devices such as guide wires and central venous catheters into a blood vessel. For example, a central venous line may be inserted into the subclavian vein for the purpose of infusing fluid intravenously over a long period. The sheath 50 thus takes the form and function of an introducer sheath and may be formed in any convenient way.

It may be preferred to have a hub and sheath which can be separated into two halves by either peeling apart long axially oriented weakened sections, or by cutting, or a combination of both so that they can be removed after a long intravenous catheter has been threaded through them.

A particular variation of the sheath 50 for the purpose of guiding large devices into blood vessels is that the sheath 50 may be tapered so that the proximal opening is larger than the distal opening. This sheath 50, when in a vein or artery, may be used as a port for the insertion of a catheter, guidewire or endoscopic device.

The sleeve 28 may also be tapered at least in its outside diameter so that it forms a fairly close fit with the proximal end of sheath 50. In this way, when the hub 18, with attached needle 12 and sleeve 28, now in the second position as shown in FIG. 4 is withdrawn from the sheath 50 and the tissue as shown in FIG. 5, an adequate opening is presented to the operator for the introduction of large intravenous devices through the sheath 50.

In accordance with the present invention, a tubular needle having an outer sleeve may be constructed such that partial or complete withdrawal of the needle from living body tissue has the effect of automatically enclosing the sharp point of the needle 12, so preventing needlestick injury.

An important feature of the present invention is that no unusual or special action is required of the operator who uses the invention. No trigger is required to make the apparatus of the present invention work. For certain purposes however it may be desirable to allow the operator to exercise some control over when the automatic sleeved needle moves from the tissue-piercing first position to the safe second position. Such control may be provided by small modifications to the concept if desired, for example by providing an outwardly extending button formed on one or more of the fingers 36 passing outwardly through the hub 18. Thus, means can be provided allowing the operator to prevent movement to the second position, such movement being prevented by finger tip pressure on the button. Such control may be appropriate when the needle is used to access a subcutaneous vein.

An important feature of the present invention is that in the action of removal of the needle from the living tissue the needle 12 moves to the position shown in FIG. 2 and FIG. 4 after which it is unable to pierce tissue a second time. However, it is possible that an attempt can be made to re-use the needle apparatus 10 by cutting off the sleeve 28. To defeat such re-use attempts the needle 12 may incorporate one or more holes or slots or be constituted of C-section tube so that in the absence of at least the part of the sleeve 28 the needle will not, in its entirety, provide a leakfree fluid path.

Modifications and variations as would be apparent to a skilled addressee are deemed to be within the scope of the present invention. For example, the or each finger 36 could be located externally of the hub 18 as could the projection 34.

Further, the proximal end of the sleeve 28 may be made of harder material than the part which enters the tissue with the needle 12. This harder proximal part might extend distally out of the hub 18.

One way to do this would be to form a forward extension on the flange and attached the soft part of the sleeve to this forward extension. The forward extension would be of harder material than the sleeve. The free travel of the flange and forward extension would be long enough to bring the forward extension just distal to the needle tip 14 as the needle 12 is withdrawn from the tissue. The used needle tip 14 would then be enclosed in a fairly hard tube with the soft sleeve dangling beyond it.

Such an arrangement could be very useful for insulin syringes used by diabetics for example. The hard proximal sleeve or extension on the projection 34 could be metal and be thin enough to penetrate tissue or it could be or larger diameter so that it stops at the skin surface.

1-15. (canceled)

16. A needle apparatus wherein a thin walled sleeve having a proximal end and a distal end, a tubular needle having a distal end and a proximal end, the needle having a sharp point at the distal end thereof, the sleeve being mounted about the needle and the needle being closely engaged by the sleeve, the needle and the sleeve being longitudinally moveable relative to one another between a first position at which the needle extends from the sleeve and a second position at which the sharp point is located within the sleeve, the needle remaining at least partially within the sleeve at all times, the apparatus further comprising a hub in which the needle is fixedly mounted adjacent the proximal end of the needle such that the needle and the hub are arranged to move longitudinally together at all times, the needle extending from the hub so that the distal end of the needle is located externally of the hub, the sleeve having the proximal end thereof located within the hub and being longitudinally slidably mounted relative to the hub, the sleeve extending from the hub so that the distal end thereof is located externally of the hub, the sleeve being unlatched in the first position but being latched in the second position, the needle being arranged to pierce tissue when the needle and the sleeve are in the first position to form an incision and the sleeve being arranged to enter the incision such that after the tissue has been pierced the needle is arranged to be withdrawn from the tissue by application to the hub of longitudinal traction in the proximal direction by an operator whilst the sleeve remains in situ in the tissue as a result of pressure applied radially by surrounding tissue, the arrangement being such that when the needle has been withdrawn to the second position and the sleeve is latched the sleeve is prevented from being retracted relative to the needle.

17. A needle apparatus according to claim 16, wherein subsequent to withdrawal of the needle from the tissue the sleeve is arranged to be withdrawn from the tissue by application of further longitudinal traction to the hub.

18. A needle apparatus according to claim 16, wherein the sleeve is provided with a laterally extending projection and means is provided for positively engaging the projection in the second position so as to latch the sleeve.

19. A needle apparatus according to claim 18, wherein the projection is located within the hub.

20. A needle apparatus according to claim 18, wherein the projection is in the form of a disc.

21. A needle apparatus according to claim 18, wherein there is provided at least one finger arranged to engage with the projection in the second position so as to prevent movement of the sleeve towards the proximal end thereof.

22. A needle apparatus according to claim 21, wherein the or each finger is located within the hub.

23. A needle apparatus according to claim 16, wherein the needle moves a short distance between the first and second positions.

24. A needle apparatus according to claim 16, wherein the needle remains in the fluid flow path of the apparatus at all times.

25. A needle apparatus according to claim 16, wherein the pressure applied by the surrounding tissue acts directly on the sleeve.

26. A method operating a needle apparatus according to claim 16, wherein tissue is pierced by the sharp point of the needle with the needle and the sleeve in the relative first position, the sleeve then enters the tissue and the needle is subsequently withdrawn from the tissue by application of longitudinal traction to the hub by an operator in the proximal direction until the needle and the sleeve reach the second relative position.

27. A method according to claim 26, wherein following withdrawal of the needle from the tissue the sleeve may be withdrawn from the tissue by application of further longitudinal traction to the hub.

28. A needle apparatus wherein a thin walled sleeve having a proximal end and a distal end, a tubular needle having a distal end and a proximal end, the needle having a sharp point at the distal end thereof, the sleeve being mounted about the needle and the needle being closely engaged by the sleeve, the needle and the sleeve being longitudinally moveable relative to one another between a first position at which the needle extends from the sleeve and a second position at which the sharp point is located within the sleeve, the needle remaining at least partially within the sleeve at all times, the apparatus further comprising a hub in which the needle is fixedly mounted adjacent the proximal end of the needle such that the needle and the hub are arranged to move longitudinally together at all times, the needle extending from the hub so that the distal end of the needle is located externally of the hub, the sleeve having the proximal end thereof located within the hub and being longitudinally slidably mounted relative to the hub, the sleeve extending from the hub so that the distal end thereof is located externally of the hub, the sleeve being unlatched in the first position but being latched in the second position, the needle being arranged to pierce tissue when the needle and the sleeve are in the first position to form an incision and the sleeve being arranged to enter the incision, a catheter introducer being mounted about the sleeve initially so as to frictionally engage the sleeve, the catheter introducer comprising a sheath which enters the tissue simultaneously with the sleeve, such that after the tissue has been pierced the needle is arranged to be withdrawn from the tissue by application to the hub of longitudinal traction in the proximal direction by an operator whilst the sleeve and the sheath remain in situ in the tissue as a result of the pressure applied radially by surrounding tissue to the sheath, the arrangement being such that when the needle has been withdrawn to the second position and the sleeve is latched the sleeve is prevented from being retracted relative to the needle.

29. A needle apparatus according to claim 28, wherein when the sleeve moves to the second position the sleeve may be withdrawn from the sheath to leave the catheter introducer in place in the tissue.

30. A needle apparatus according to claim 29, wherein the catheter introducer also has a further hub attached to the sheath, the further hub being arranged to be restrained from movement manually or by attachment to skin upon movement of the sleeve.

31. A needle apparatus according to claim 28, wherein the catheter introducer after withdrawal of the needle apparatus is arranged to act as a guide for introduction of longer devices such as guide wires, catheters and endoscopic devices.
